(19) United States
(12) Patent Application Publication
(10) Pub. No.: US 2026/0186128 A1
(43) Pub. Date: Jul. 2, 2026

(54) mmWave Radar System for Ambient Sensing

(71) Applicant: KaiKuTek INC., Taipei (TW)

(72) Inventors: Tsung-Ming Tai, Taipei (TW); Mike Chun-Hung Wang, Taipei (TW); Wen-Jyi Hwang, Taipei (TW); Wei-Chi Li, Taipei (TW); Kuan-Yu Liu, Taipei (TW)

(73) Assignee: KaiKuTek INC., Taipei (TW)

(21) Appl. No.: 19/006,246

(22) Filed: Dec. 31, 2024

(51) Int. Cl. G01S 13/89; A61B 5/0507; G01S 13/10

(52) U.S. Cl. CPC G01S 13/89; A61B 5/0507; G01S 13/10

(57) ABSTRACT

An mmWave radar system includes an mmWave radar sensor, an interactive device, and a control unit. The mmWave radar sensor is used to detect environmental data. The control unit is coupled to the mmWave radar sensor and the interactive device, and used to process the environmental data, and operate the interactive device according to the environmental data.

### BACKGROUND OF THE INVENTION

### 1. Field of the Invention

The present invention relates to an mmWave radar system, in particularly relates to an mmWave radar system for ambient sensing.

### 2. Description of the Prior Art

Ambient sensing is sensing technology that gathers data about one's surroundings. This data can be used for a variety of purposes, such as understanding the environment, providing information to users, or controlling devices.

In ambient sensing, a whole set of sensors work together to provide information. While, in principle, nearly any sensor could be calibrated as an ambient sensor, some of the most common forms of ambient sensing include temperature sensors, pressure sensors, water sensors, and object sensors.

Temperature sensors can keep a constant check on temperatures within the home. Average temperatures could be used to provide indications to whether the home remains at a healthy temperature or not. Pressure sensors can work with temperature sensors to form a picture of weather conditions around the home. Water sensors can provide information about increased humidity in the home. Object sensors (with RFID tags or GPS trackers) might be placed on key items, or individuals to draw general insights on use and movement.

However, conventional ambient sensing or environmental sensors have limited utility due to their simple outputs.

### SUMMARY OF THE INVENTION

An embodiment provides an mmWave radar system. The mmWave radar system includes an mmWave radar sensor, an interactive device, and a control unit. The mmWave radar sensor is used to detect environmental data. The control unit is coupled to the mmWave radar sensor and the interactive device, and used to process the environmental data, and operate the interactive device according to the environmental data.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a block diagram of an mmWave radar system according to an embodiment of the present invention.

FIG. 2 is a schematic diagram of the mmWave radar system in FIG. 1 according to an embodiment of the present invention.

FIG. 3 is a schematic diagram of the mmWave radar system in FIG. 1 with responses to the ambient sensing according to another embodiment of the present invention.

### DETAILED DESCRIPTION

Millimeter wave (mmWave) sensing is a non-contact technology that uses mmWave radar sensors to measure movement, acceleration, and angles with precision down to a fraction of a millimeter. This system operates by transmitting and receiving pulses of millimeter electromagnetic wave energy, detecting targets and motion from the reflected signals. Additional components such as converters, signal processors, and other embedded technologies enhance the system's performance and enable new applications. Current uses of this technology include tracking human and animal movement, detecting human presence, and monitoring vital signs. These applications span various industries, including automotive, meteorological, medical, and pet health, often serving as an alternative to wearable-based technologies.

Compared to other radio frequency sensing technologies in the electromagnetic spectrum, such as infrared or ultra-wideband, mmWave operates in the 10 to 100 gigahertz (GHz) range. Typical mmWave sensors utilize the 24 GHz, 60 GHz, and 77 GHz bands, each offering unique advantages for specific applications.

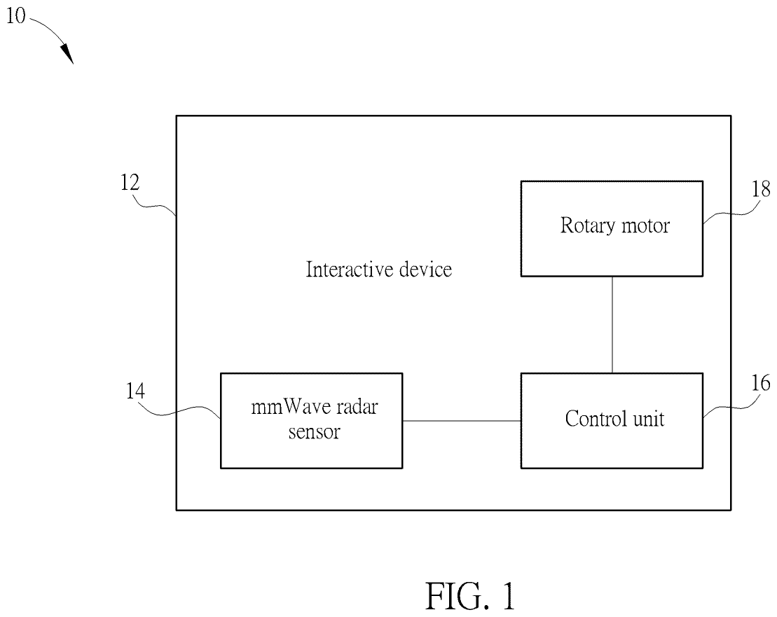

FIG. 1 is a block diagram of an mmWave radar system 10 according to an embodiment of the present invention. The mmWave radar system 10 may comprise an interactive device 12, an mmWave radar sensor 14, a control unit 16 and a rotary motor 18. The mmWave radar sensor 14, a control unit 16 and a rotary motor 18 may be disposed inside the interactive device 12. The control unit 16 may be coupled to the mmWave radar sensor 14, the rotary motor 18, and the interactive device 12. The mmWave radar sensor 14 is used to detect environmental data, and the control unit 16 is used to process the environmental data and operate the interactive device 12 according to the environmental data. In an embodiment, the environmental data includes heart rate, respiratory rate, gesture, posture, position and/or velocity of a living being. In another embodiment, the environmental data includes position and/or velocity of a non-living object.

In an embodiment, the control unit 16 adjusts the rotary motor 18 according to the environmental data to change an orientation of the interactive device 12 according to the environmental data. For example, the interactive device 12 is a smart fan, and the mmWave radar sensor 14 detects the position of a person. The control unit 16 may adjust the rotary motor 18 to orient the smart fan towards the person based on the detected position. In another example, the interactive device 12 is a spatial audio, and the mmWave radar sensor 14 detects the position of a person. The control unit 16 may adjust the spatial audio to ensure it surrounds the person based on the detected position. In another embodiment, the mmWave radar sensor 14 detects the position of a non-living object, and the control unit 16 may adjust the interactive device 12 according to the position of the non-living object.

In an embodiment, The mmWave radar system 10 may comprise the interactive device 12, the mmWave radar sensor 14 and the control unit 16 without the rotary motor 18. The mmWave radar sensor 14 and the control unit 16 may be disposed inside the interactive device 12. The control unit 16 may be coupled to the mmWave radar sensor 14 and the interactive device 12. In an embodiment, the control unit 16 turns on or off the interactive device 12 according to the environmental data. For example, the interactive device 12 is a smart television, and the mmWave radar sensor 14 detects the gesture or posture of a person. The control unit 16 may adjust or turn on/off the smart television according to the detected gesture or detected posture of the person. In another example, the interactive device 12 is an electric door, and the mmWave radar sensor 14 detects the gesture or posture of a person. The control unit 16 may open or close the electric door according to the detected gesture or detected posture of the person. In another example, the interactive device 12 is an air conditioner, and the mmWave radar sensor 14 detects the gesture or posture of a person. The control unit 16 may adjust or turn on/off the air conditioner according to the detected gesture or detected posture of the person. In another embodiment, the mmWave radar sensor 14 detects the position and/or movement of a non-living object, and the control unit 16 may turn on/off the interactive device 12 according to the position and/or movement of the non-living object.

In an embodiment, the environmental data includes heart rate, respiratory rate, gesture, posture, position and/or velocity of a living being. Therefore, the mmWave radar sensor 14 may detect the presence, location, movement, gesture, and/or posture of a person. The control unit 16 can adjust the interactive device 12 based on the detected presence, location, movement, gesture, and/or posture from the mmWave radar sensor 14.

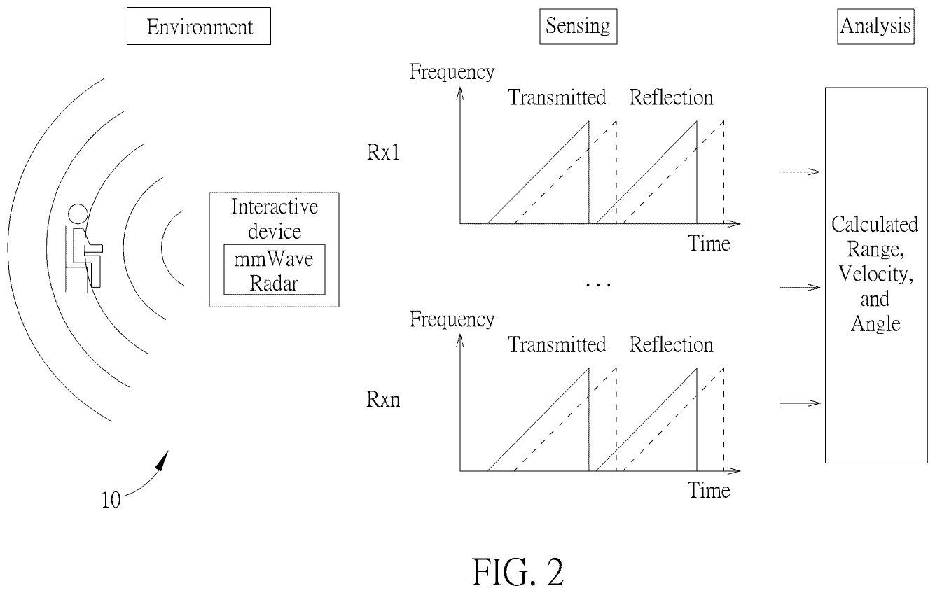

FIG. 2 is a schematic diagram of the mmWave radar system 10 according to an embodiment of the present invention. The mmWave radar system 10 includes n transmitters and n receivers Rx1 to Rxn. In an embodiment, the n transmitters transmit a frequency modulated continuous wave (FMCW) signal, and the FMCW signal is reflected from a target (for example, a human being) to generate a reflected FMCW signal. Then, the reflected FMCW signal is received by the plurality of receivers Rx1 to Rxn to generate n received FMCW signals.

The received FMCW signals are delayed compared to the FMCW signal, thereby the range of the target can be analyzed by calculating the frequency difference between each of the received FMCW signals and the FMCW signal. The range of the target refers to the distance between the target and the mmWave radar system 10. The phase difference among chirps of the received FMCW signals may be analyzed to generate the velocity of the target, and the phase differences among different receivers Rx1 to Rxn may be analyzed to generate the angle of arrival (AoA) of the target. The AoA is an angle from the target to the platform of the antenna array of the mmWave radar system 10. The antenna array may include n antennas used for n transmitters and n receivers. In an embodiment, the range, velocity and angle can be calculated using the transmitting and receiving of the FMCW signal with the antenna array of the mmWave radar system 10. In another embodiment, the mmWave radar system 10 transmits and receives pulses to generate the range, velocity and angle of the target.

In an embodiment, the environmental data includes heart rate, respiratory rate, gesture, posture, position and/or velocity of a living being. The control unit 16 can adjust the interactive device 12 based on the detected presence, location, movement, gesture, and/or posture using a machine learning model. The machine learning model is trained according to the environmental data and the analyzed result of the environmental data. The machine learning model can apply to ambient sensing based on the environmental data received by the mmWave radar sensor 14.

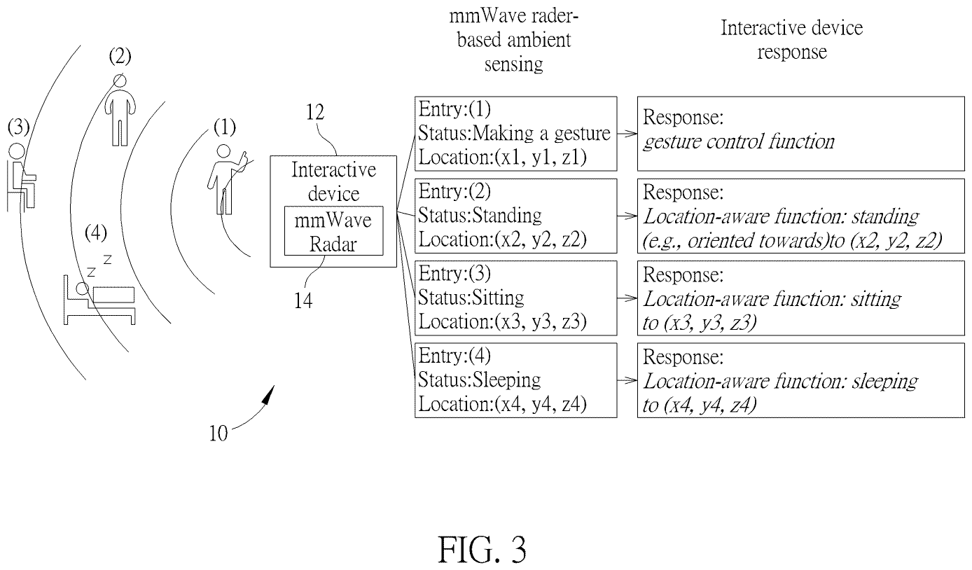

FIG. 3 is a schematic diagram of the mmWave radar system 10 with responses to the ambient sensing according to another embodiment of the present invention. In FIG. 3, there are four people in the environment. Person 1 is making a gesture, person 2 is standing, person 3 is sitting, and person 4 is sleeping. The mmWave radar sensor 14 detects the environmental data from people 1 to 4. The analyzed results may include person 1 making a gesture at location (x1, y1, z1), person 2 standing at location (x2, y2, z2), person 3 sitting at location (x3, y3, z3), and person 4 sleeping at location (x4, y4, z4). The control unit 16 adjusts the interactive device 12 to respond to the analyzed results. The gesture made by person 1 at location (x1, y1, z1) may trigger the control of a smart television, air conditioner, electric door, smart lighting, security surveillance system, and/or another smart device. The person 2 standing at location (x2, y2, z2) may be detected by the mmWave radar sensor 14, and the control unit 16 may adjust a smart fan, air conditioner, spatial audio and/or another smart device accordingly. The person 3 sitting at location (x3, y3, z3) may be detected by the mmWave radar sensor 14, and the control unit 16 may adjust a smart fan, air conditioner, spatial audio and/or another smart device accordingly. The person 4 sleeping at location (x4, y4, z4) may be detected by the mmWave radar sensor 14, and the control unit 16 may adjust a smart fan, air conditioner, spatial audio and/or another smart device accordingly. In an embodiment, the four people 1 to 4 may be detected by an mmWave radar system 10 or by a plurality of mmWave radar systems to adjust the interactive device 12 or a plurality of interactive devices. The number of mmWave radar system 10 and the number of the interactive device 12 can be any integer and are not limited in the present invention.

In summary, the mmWave radar system 10 detects environmental data and processes the environmental data to adjust the interactive device 12 according to the environmental data. The mmWave radar sensor 14 may detect the heart rate, the respiratory rate, the presence, the position, the movement, the gesture, and/or the posture of a living object, thereby the interactive device can respond to the presence and behavior of living object accordingly.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An mmWave radar system, comprising:
an mmWave radar sensor, configured to detect environmental data;
an interactive device; and
a control unit, coupled to the mmWave radar sensor and the interactive device, and configured to process the environmental data, and operate the interactive device according to the environmental data.

2. The mmWave radar system of claim 1, wherein the environmental data comprises heart rate, respiratory rate, gesture, posture, position and/or velocity of a living being.

3. The mmWave radar system of claim 1, wherein the environmental data comprises position and/or velocity of a non-living object.

4. The mmWave radar system of claim 1, wherein the interactive device comprises a rotary motor.

5. The mmWave radar system of claim 4, wherein the control unit adjusts the rotary motor according to the environmental data.

6. The mmWave radar system of claim 5, wherein the rotary motor is adjusted to change an orientation of the interactive device according to the environmental data.

7. The mmWave radar system of claim 6, wherein the environmental data comprises heart rate, respiratory rate, gesture, posture, position and/or velocity of a living being.

8. The mmWave radar system of claim 6, wherein the environmental data comprises position and/or velocity of a non-living object.

9. The mmWave radar system of claim 1, wherein the control unit turns on or off the interactive device according to the environmental data.

10. The mmWave radar system of claim 9, wherein the environmental data comprises heart rate, respiratory rate, gesture, posture, position and/or velocity of a living being.

11. The mmWave radar system of claim 9, wherein the environmental data comprises position and/or velocity of a non-living object.

12. The mmWave radar system of claim 1, wherein the interactive device is a smart fan, a smart television, a spatial audio, an air conditioner, smart lighting, security surveillance system, or an electric door.

13. The mmWave radar system of claim 1, wherein the control unit processes the environmental data, and operates the interactive device according to the environmental data based on a machine learning model.

14. The mmWave radar system of claim 1, wherein the mmWave radar sensor transmits a plurality of frequency modulated continuous wave (FMCW) signals and receives a plurality of reflected FMCW signals.

15. The mmWave radar system of claim 14, wherein the control unit generates range, velocity, and angular position of an object according to the reflected FMCW signals.
